(19) United States
(12) Patent Application Publication
(10) Pub. No.: US 2007/0270960 A1
(43) Pub. Date: Nov. 22, 2007

(54) Extendable anchor in a vertebral implant and methods of use

(75) Inventors: Henry Keith Bonin JR., Memphis, TN (US); Jason A. Edie, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/409,678

(22) Filed: Apr. 24, 2006

(51) Int. Cl. A61F 2/44

(52) U.S. Cl. 623/17.11

(57) ABSTRACT

A vertebral implant includes a body including an outer surface and an aperture forming an inner cavity. A gear including a plurality of teeth is rotatable within the cavity about a rotation axis. An anchor including a bone-engaging feature to engage the vertebral members may be operatively coupled to the gear. The gear teeth may be accessible from outside the body to rotate the gear. The anchor may be movable substantially along the rotation axis between a first position in which the bone-engaging feature is retracted below the outer surface and a second position in which the bone-engaging feature is extended above the outer surface.

### BACKGROUND

Vertebral implants are often used in the surgical treatment of spinal disorders such as degenerative disc disease, disc herniations, curvature abnormalities, and trauma. Many different types of treatments are used. In some cases, spinal fusion is indicated to inhibit relative motion between vertebral bodies. In other cases, dynamic implants are used to preserve and/or restore motion between vertebral bodies. Further, many different types of implants are used, including for example, rods, plates, spacers, and interbody implants. Conventionally, interbody implants are inserted into the space between vertebral bodies after the intervertebral disc material has been removed. Interbody implants may include one or more anchor members that extend beyond superior and/or inferior surfaces of the implant that are configured to engage or embed within a vertebral body. The anchors may enhance the stability of the implant by improving the connection between the implant and the adjacent vertebral bodies. The anchors may also establish a permanent orientation of the device in the intervertebral disc space.

Unfortunately, because the anchors protrude beyond the superior and/or inferior surfaces of the implant, the anchors tend to increase the height of the implant. During insertion, implants with a greater height sometimes require greater distraction of the vertebral bodies between which the implant is placed. Consequently, the advantages offered by the height of the anchors tend to be offset by the risk of over-distraction of the spine during implant insertion.

### SUMMARY

Illustrative embodiments disclosed herein are directed to a vertebral implant that includes a body with an outer surface and an aperture forming an inner cavity. A gear including a plurality of teeth is rotatable within the cavity about a rotation axis. In one embodiment, the aperture in the body and the outer perimeter of the gear teeth include mating threads. An anchor including a bone-engaging feature to engage the vertebral members may be operatively coupled to the gear. In one embodiment, the anchor is coupled to the gear. In one embodiment the anchor is disposed in sliding contact with the gear. The gear teeth may be accessible from outside the body to rotate the gear. The anchor may be movable substantially along the rotation axis between a first position in which the bone-engaging feature is retracted below the outer surface and a second position in which the bone-engaging feature is extended above the outer surface. As the gear is rotated, the gear may also move within the threaded aperture to push the anchor. The gear teeth may be accessible through the lateral opening regardless of the position of the gear within the aperture.

### BRIEF DESCRIPTION OF THE DRAWINGS

FIGS. 1A and 1B are side views of a vertebral implant according to one embodiment shown relative to vertebral bodies;

FIGS. 2A and 2B are schematic diagrams illustrating functional components in a vertebral implant according to one or more embodiments;

FIG. 3 is an exploded perspective assembly view of a vertebral implant according to one embodiment;

FIG. 4 is a lateral view of a vertebral implant and an installation tool according to one embodiment;

FIGS. 5A, 5B, and 5C are perspective views of a vertebral implant according to one embodiment and depict a sequence in which an anchor member proceeds between a retracted position, an intermediate position, and an extended position;

FIGS. 6A and 6B are perspective section views of a vertebral implant according to one embodiment and depict a sequence in which an anchor member proceeds between a retracted position and an extended position;

FIG. 7 is a top section view of a vertebral implant including a locking mechanism according to one embodiment;

FIG. 8 is a top section view of a vertebral implant including a locking mechanism according to one embodiment;

FIG. 9 is a top view of components of one embodiment of a vertebral implant in an unassembled state; and

FIG. 10 is a side section view of a vertebral implant according to one embodiment.

### DETAILED DESCRIPTION

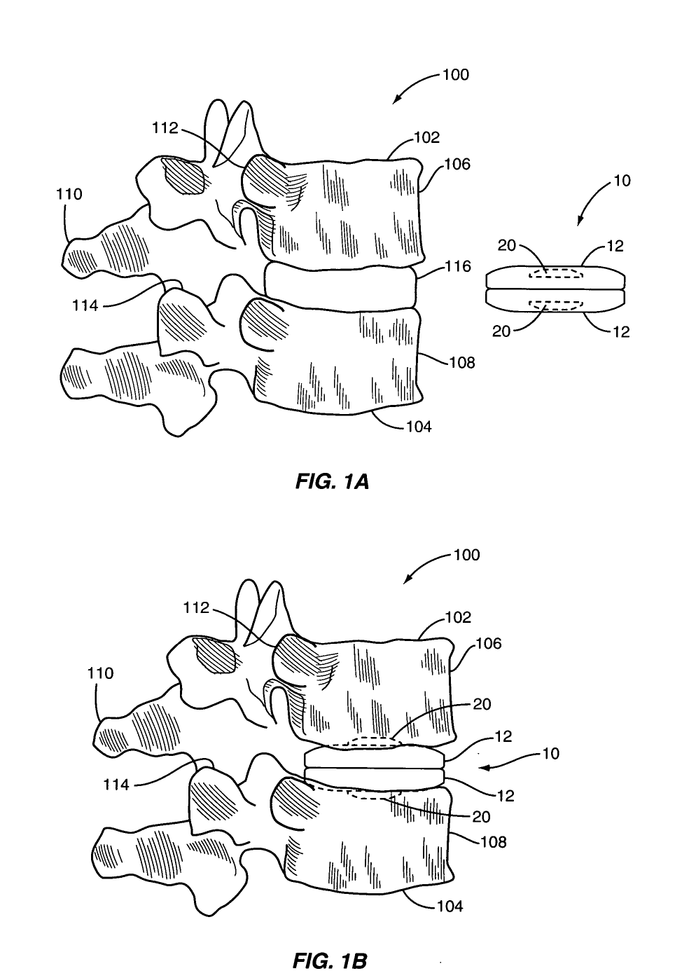

The various embodiments disclosed herein relate to a vertebral implant including a retractable anchor member. The anchor member may be retracted to decrease the overall height of the implant for insertion. Once the implant is positioned as desired, the anchor may be extended to engage the vertebral body against which the implant is placed. Reference number 10 in FIGS. 1A and 1B generally identifies one example of an implant with the retractable anchor member. The representative vertebral implant 10 is illustrated as a disc replacement implant that is inserted between vertebral bodies of a patient as part of a disc replacement surgery. FIGS. 1A and 1B show two vertebrae 102, 104 and a disc 116 therebetween. Each vertebra 102, 104 includes a generally cylindrical body 106, 108 that contributes to the primary weight-bearing portion of the spine 100. Further, each vertebra 102, 104 includes various bony processes 110, 112 extending posterior to the body 106, 108. Adjacent vertebrae 102, 104 may move relative to each other via facet joints 114 and due to the flexibility of the disc 116. For instances where the disc 116 is herniated or diseased, or other conditions exist, the entire disc 116 may be replaced with the vertebral implant 10 using an anterior approach as shown. The vertebral implant 10 may be inserted from a lateral approach, which may eliminate the need to remove surrounding tissue, such as the anterior longitudinal ligament, thus contributing to the overall stability of the motion segment. In other implementations, the vertebral implant 10 may be inserted from a posterior approach.

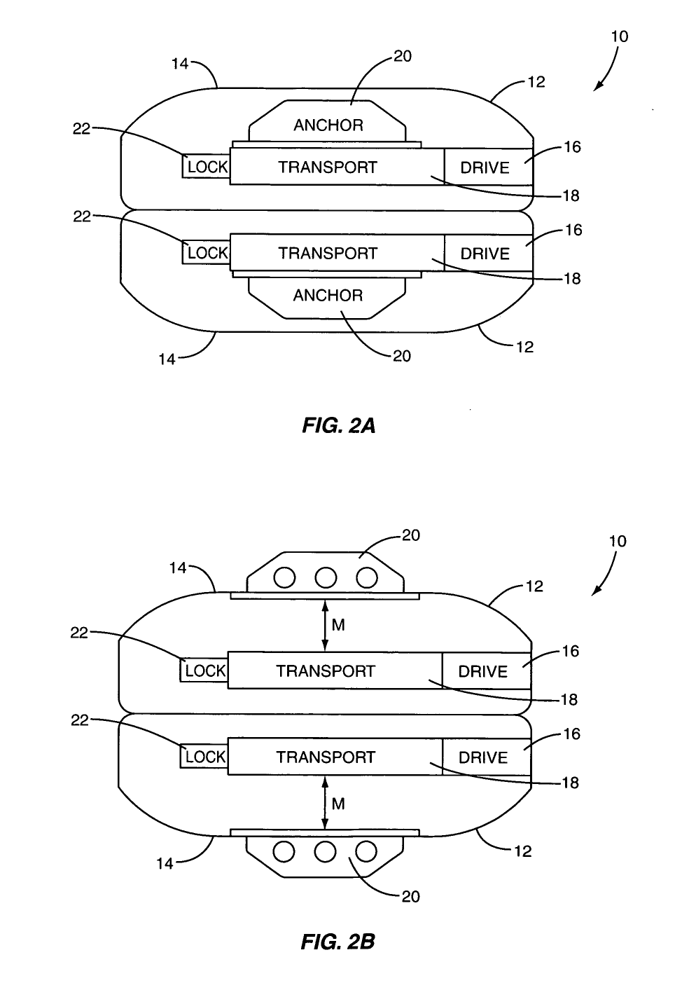

In the illustrated embodiment, the vertebral implant 10 includes two implant bodies 12 that are arranged in a stacked configuration. Each implant body 12 includes a retractable anchor member 20 contained therein and identified by dashed lines. The vertebral implant 10 is insertable between the vertebral bodies 106, 108 while the anchor members 20 are retracted. Once the vertebral implant 10 is inserted between the vertebral bodies 106, 108, the anchor members 20 may be extended to engage or embed within the vertebral bodies 106, 108 as shown in FIG. 1B. Various techniques for extending the anchor members 20 are discussed in detail below. For now, the schematic representations of the vertebral implant 10 shown in FIGS. 2A and 2B depict the general function and operation of the vertebral implant 10.

FIGS. 2A and 2B depict a schematic representation of various functional components within the vertebral implant 10. FIG. 2A portrays the anchor members 20 in a retracted position, contained within each of the upper and lower implant bodies 12. In contrast, FIG. 2B portrays the anchor members 20 in an extended position, protruding beyond respective bone contact surfaces 14 of the implant bodies 12.

In the embodiment shown, each anchor member 20 includes a corresponding drive mechanism 16, transport mechanism 18, and lock mechanism 22. The transport mechanism 18 provides the moving force that translates the anchor member 20 in the direction indicated by the arrows labeled M in FIG. 2B. The drive mechanism 16 provides an interface by which a surgeon may engage the transport mechanism 18 to extend or retract the anchor members 20. Some non-limiting examples of transport mechanisms 18 may include pneumatic or electric transducers, expandable members, and threaded assemblies. In one embodiment, the transport mechanism 18 may be provided by a cam surface or tapered surface that imparts motion to the anchor member 20 in the extension direction upon introducing an input force from a different input direction. In one or more embodiments, the drive mechanism 16 provides the transport mechanism 18 with enough input force to move the coupled anchor member 20 with enough force to penetrate the vertebral body endplates and cortical shell.

A surgical tool that includes, for example, a driver, a wrench, a gear, or means to release a switch can engage the drive mechanism 16 to actuate the transport mechanism 18. For instance, a surgical tool may have a mechanical driver that engages a mating feature in the drive mechanism 16. Some conventionally known mechanical drive features include hex shaped bodies or recesses, gear teeth, and apertures such as those engaged by a spanner wrench.

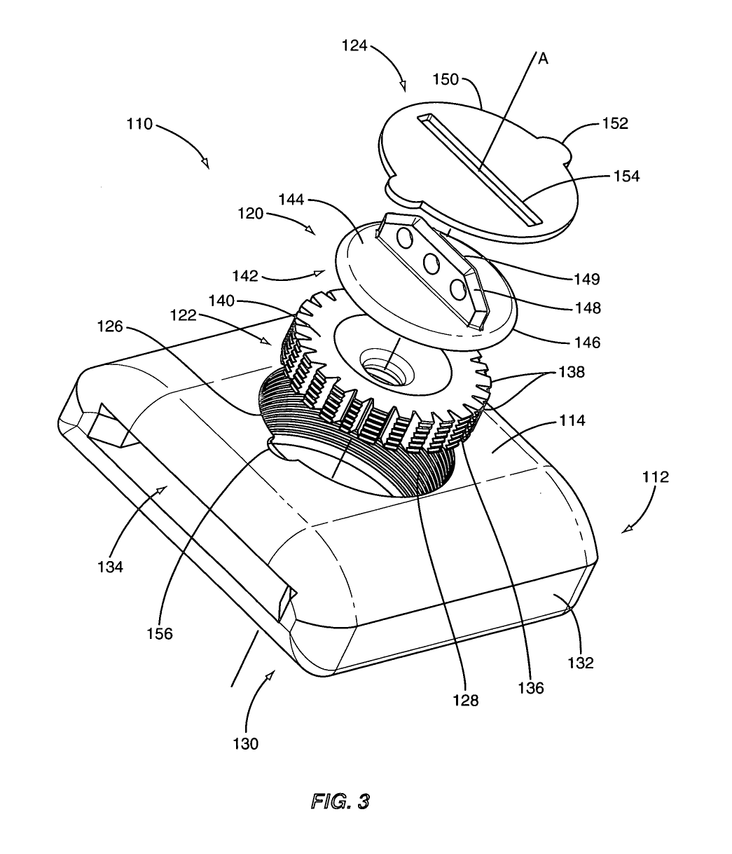

In one embodiment, the drive mechanism 16 is implemented as an input gear while the transport mechanism 18 is implemented as a threaded feature that extends or retracts the anchor member 20 upon rotating the input gear. FIG. 3 illustrates one embodiment of a vertebral implant 110 incorporating a drive mechanism 16 and transport mechanism 18 of this type. Specifically, FIG. 3 illustrates an exploded assembly view of a vertebral implant 110 that includes a body 112, an anchor member 120, a gear member 122, and a cover 124.

The body 112 of the vertebral implant 110 shown in FIG. 3 includes a simple rectangular shape, though other shapes and contours may be used. In further embodiments, the vertebral implant 110 may take on other types of configurations, such as, for example, a circular shape, kidney shape, semi-oval shape, bean-shape, D-shape, elliptical-shape, egg-shape, or any other shape that would occur to one of skill in the art. The vertebral implant 110 may take on substantially solid configurations, such as, for example, block-like or plate-like configurations that do not define an open inner region. In other embodiments, the vertebral implant 110 could also be described as being annular, U-shaped, C-shaped, V-shaped, horseshoe-shaped, semi-circular shaped, semi-oval shaped, or other similar terms defining an implant including at least a partially open or hollow construction.

The vertebral implant 110 may be constructed from biocompatible metal alloys such as titanium, cobalt-chrome, and stainless steel. The vertebral implant 10 may be constructed from non-metallic materials, including for example, ceramics, resins, or polymers, such as UHMWPE and implantable grade polyetheretherketone (PEEK) or other similar materials (e.g., PAEK, PEKK, and PEK). The vertebral implant 10 may be constructed of synthetic or natural bone or bone composites. Those skilled in the art will comprehend a variety of other material choices that are suitable for the illustrated vertebral implant 10.

The body 112 of the vertebral implant 110 includes a bone-contact surface 114 and an opposite surface 130, which is disposed at a bottom side of the body 112 in the orientation shown in FIG. 3. Further, the body 112 includes a sidewall 132 extending about the perimeter of the body 112 and extending between the bone-contact surface 114 and opposite surface 130. The bone-contact surface 114 includes an aperture 126 sized to accept the anchor member 120, a gear member 122, and a cover 124. In the embodiment shown, the aperture 126 extends from the bone-contact surface 114 into the body 112 and may extend through the opposite surface 130, though this is not required. That is, the aperture 126 may be a blind hole that includes a bottom surface (not shown).

In the exemplary embodiment, the outer wall 128 of the aperture 126 includes a female thread. A corresponding male thread 136 is included at the perimeter of the gear member 122 so that the gear member 122 may be screwed into or out of the aperture 126. The gear member 122 also includes a plurality of gear teeth 138 disposed about the perimeter. The threads 136 on the gear member 122 are formed on the gear teeth 138, with the voids between adjacent gear teeth 138 being open. Accordingly, the threads 136 are not continuous as they too are broken by the space between the gear teeth 138. However, a sufficient amount of thread 136 exists to engage the female threads 128 in the body 112.

FIG. 3 also shows an anchor member 120 disposed in contact with the gear member 122. The anchor member 120 includes a keel 148 that protrudes from a generally disc shaped body defined by a top surface 144, bottom surface 142, and outer perimeter 146. The keel 148 is generally thin and blade-like with a sharpened edge 149 configured to engage or embed within a vertebral body when the anchor member 120 is extended. The keel 148 may establish and maintain a desired orientation of the anchor member 120 and the implant 110 relative to the spine. For example, when the implant 110 is inserted, the keel 148 may be aligned with a desired anatomical plane (e.g., sagittal or coronal plane) and extended into the adjacent vertebral body to maintain the desired orientation. The anchor member 120 may include other types and shapes of bone engagement features, including for example, teeth or spikes. Further, the anchor member 120 may include more than one bone engagement feature, including a plurality of keels 148.

In the embodiment shown, the bottom surface 142 of the anchor member 120 is a bearing surface that abuts bearing surface 140 on the gear member 122. The bearing surfaces 140,142 slide relative to each other as the gear member 122 rotates about the threads 128, 136. The outer perimeter 146 of the anchor member 120 may be smaller than the minor diameter of the gear teeth 138 to prevent interference. Further, the bearing surfaces 140, 142 may include inter-engaging, centering features (not specifically illustrated) to align the members 120, 122 with each other and/or about axis A. In one embodiment, the gear member 122 and anchor member 120 are coupled to one another.

In one embodiment, the anchor member 120 is constrained to linear movement along the axis A of the threads 128, 136 because a slot 154 in the cover 124 orients the keel 148 on the anchor member 120 along a predetermined direction. That is, the keel 148 protrudes through the slot 154 in a way that prevents the anchor member 120 from rotating about axis A while permitting extension and retraction in the direction of axis A. The shape of the slot 154 in the illustrated embodiment is narrow to accommodate the blade-like shape of the keel 148. In other embodiments, the slot 154 may take on a different shape to accommodate different anchor members 120 or anchor members 120 including a plurality of bone engaging features.

The cover 124 is also constrained against rotation about axis A through engagement of tabs 152 on the cover 124 with corresponding slots 156 disposed adjacent to the aperture 126 on the implant body 112. The outer perimeter 150 of the cover 124 may be sized to substantially fill the aperture 126. Alternatively, the cover 124 may be secured over the aperture 126 and to the implant body 112 with fastening hardware such as flat head screws. Alternatively, the cover 124 may be adhered to the implant body 112 with biocompatible adhesives such as, but not limited to, PMMA. The cover 124 may also be secured to the implant body 112 by way of welding, soldering, press fitting, or other common manufacturing operations. Further, the cover 124 could be secondarily ground and polished to the implant body 112 until the two members 112, 124 essentially become a single unit.

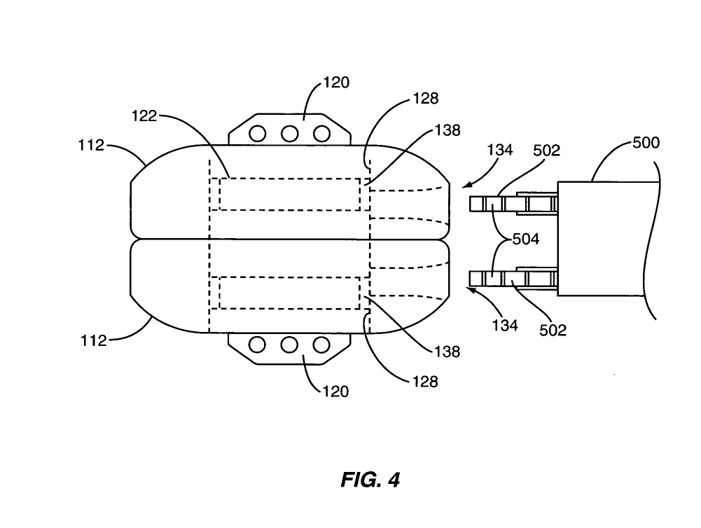

When the gear member 122 is disposed within the aperture 126, the gear teeth 138 are exposed through a lateral opening 134 in the sidewall 132. A surgeon can engage the gear teeth 138 through the lateral opening 134 using a surgical tool 500 with a rotatable gear 502 as depicted in FIG. 4. The rotatable gear 502 includes gear teeth 504 that are sized and spaced to mate with gear teeth 138. The surgical tool 500 may include an unillustrated drive train and drive source (e.g., manual, electric, pneumatic) that transmits a rotating force to the gears 502 to rotate the gear member 122. Upon rotating the gear member 122 through the lateral opening 134, the gear member 122 will move up or down depending on the direction of rotation and on the direction of the threads.

In the embodiment shown in FIG. 4, two implant bodies 112 are arranged in a stacked configuration. The two bodies 112, including the threads 128 and the mating threads 136 on the gear member 122, may be identical. Accordingly, the gears 502 on the installation tool 500 may be driven in opposite directions to extend or retract the anchor members 120 in the implant bodies 112. Those skilled in the art should comprehend that the threads 128, 136 may be formed with a conventional right hand thread in both the upper and lower bodies 112, and gear members 122. In an alternative configuration, the threads in one of the bodies 112 and gear members 122 may be right hand threads while the other body 112 and gear member 122 may include left hand threads. If the threads are formed differently in this manner, the gears 502 on the installation tool 500 may be configured to rotate in the same direction to extend or contract the anchor members 120.

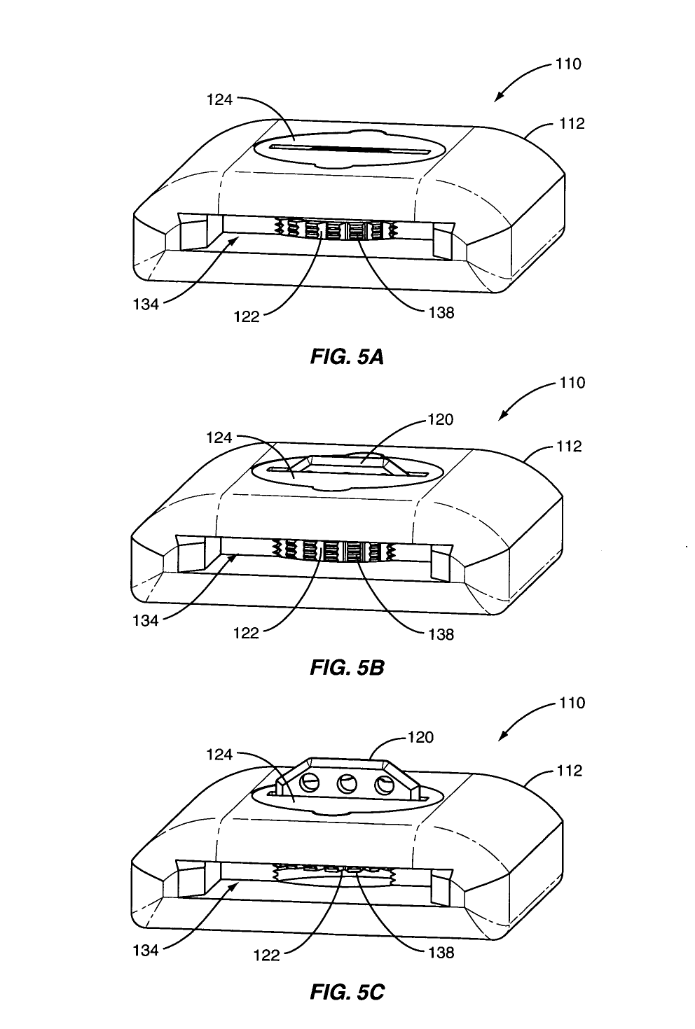

FIGS. 5A-5C show similar perspective views of the exemplary vertebral implant 110 and depict a sequence in which the gear member 122 and anchor member 120 proceed between a retracted position (FIG. 5A), an intermediate position (FIG. 5B), and an extended position (FIG. 5C). As described above, the gear teeth 138 can be engaged through the lateral opening 134. Therefore, the gear member 122 can be rotated, for example with the installation tool 500 from FIG. 4, to raise or lower the gear member 122. Since the exemplary anchor 120 contacts the gear member 122, the anchor member 120 also moves as indicated. Further, FIGS. 5A-5C show that despite the movement of the gear member 122, at least some portion of the gear teeth 138 are accessible through the lateral opening.

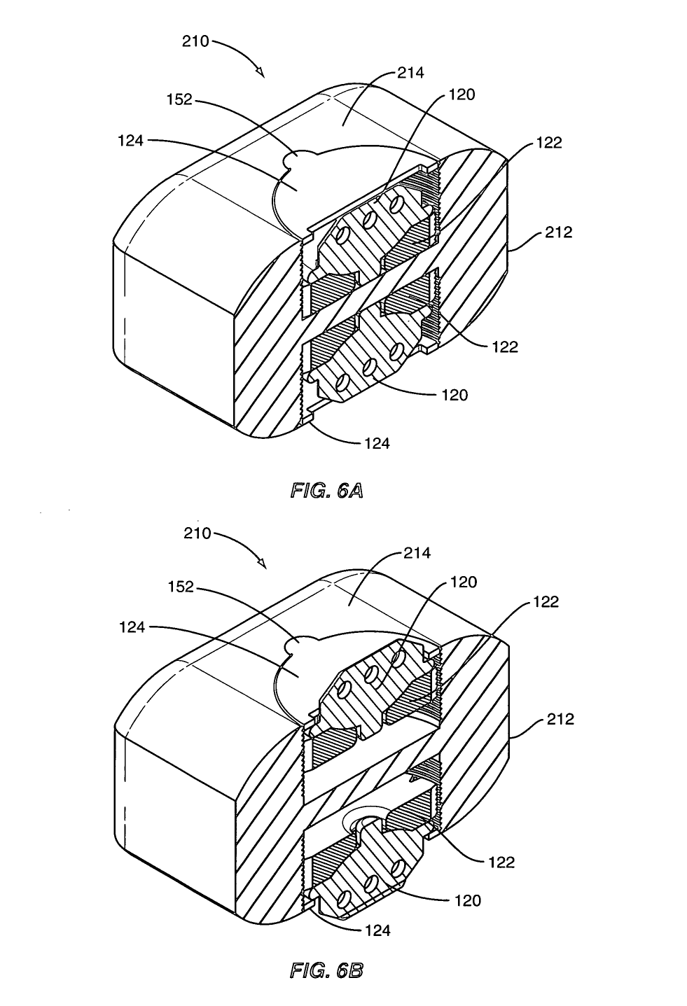

FIGS. 6A and 6B illustrate one embodiment of a vertebral implant 210 that includes a single body 212 including bone contact surfaces 214 disposed on opposite sides of the body 212. The vertebral implant 210 further includes two gear members 122, and two anchor members 120, each configured to extend outward from the bone contact surfaces 214. The vertebral implant 210 also includes a cover 124 disposed at each bone contact surface and oriented (via tabs 152) so that the anchor members 120 are substantially aligned. In other embodiments, the cover 124 may be disposed so that the anchor members 120 are other than substantially aligned. As with previous embodiments, each of the gear members 122 may be rotated to extend or retract the anchor member 120.

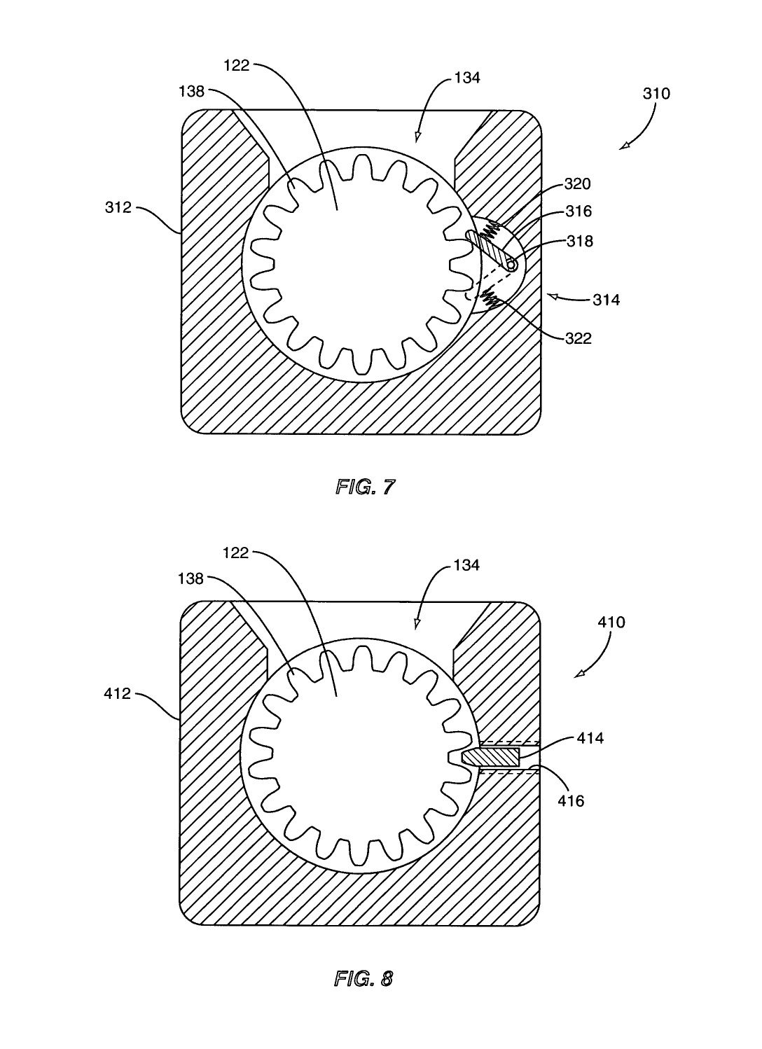

FIGS. 2A and 2B illustrated various functional components of an exemplary vertebral implant 10. A lock mechanism 22 was among the described functional components. FIGS. 7 and 8 illustrate embodiments of the vertebral implant 310, 410 that include a locking feature to secure the position of the gear member 122 and anchor 120. Each FIGS. (7 and 8) depicts a top section view of the representative vertebral implants. In FIG. 7, the body 312 includes a ratcheting mechanism 314 that is configured to allow rotation of the gear member 122 in one of two rotation directions. The ratcheting mechanism 314 includes a pivoting stopper 316 that is coupled to the body 312 at pivot 318. The end of the stopper 316 opposite the pivot 318 is urged towards the teeth 138 of the gear member 122 by a first biasing member 320. As an example, the first biasing member 320 may be a coil spring or a leaf spring. As the gear member 122 rotates in a counter-clockwise direction, the individual teeth 138 of the gear member 122 deflect the stopper 316 against the urging force provided by the first biasing member 320. Once an individual tooth 138 of the gear member 122 rotates past the stopper 316, the stopper deflects outward into the space between the teeth 138. The stopper 316 disposed in this manner prevents gear member 122 rotation in the clockwise direction. Thus, the ratcheting mechanism 314 may permit extension of an anchor member 120, but prevent retraction.

In one embodiment, the stopper 316 is selectably positioned between the solid line position and the dashed line position shown in FIG. 7. In the dashed line position, a second biasing member 322 urges the stopper 316 towards the gear member 122. Though not illustrated in FIG. 7, the stopper 316 may be selectably moveable with a switch, lever, button, or other mechanism that selects between a first position where gear member 122 motion is allowed in one direction and a second position where gear member 122 motion is allowed in another direction.

FIG. 8 depicts an embodiment of a vertebral implant 410 in which the lock function is provided by a stopper 414 that protrudes from the body 412 into the space between adjacent teeth 138 of the gear member 122. In one embodiment, as illustrated in FIG. 8, the stopper 414 may be implemented as a setscrew that is inserted through a threaded aperture 416 in the body 412 and into contact with the gear member 122. In another embodiment, the stopper 414 may be implemented as a pin, wedge, or other piece of hardware that abuts the gear member 122 to prevent rotation of the gear member 122. In another embodiment, the walls of the body 412 surrounding the gear member 122 may be compressed or staked to create an interference that prevents rotation of the gear member 122.

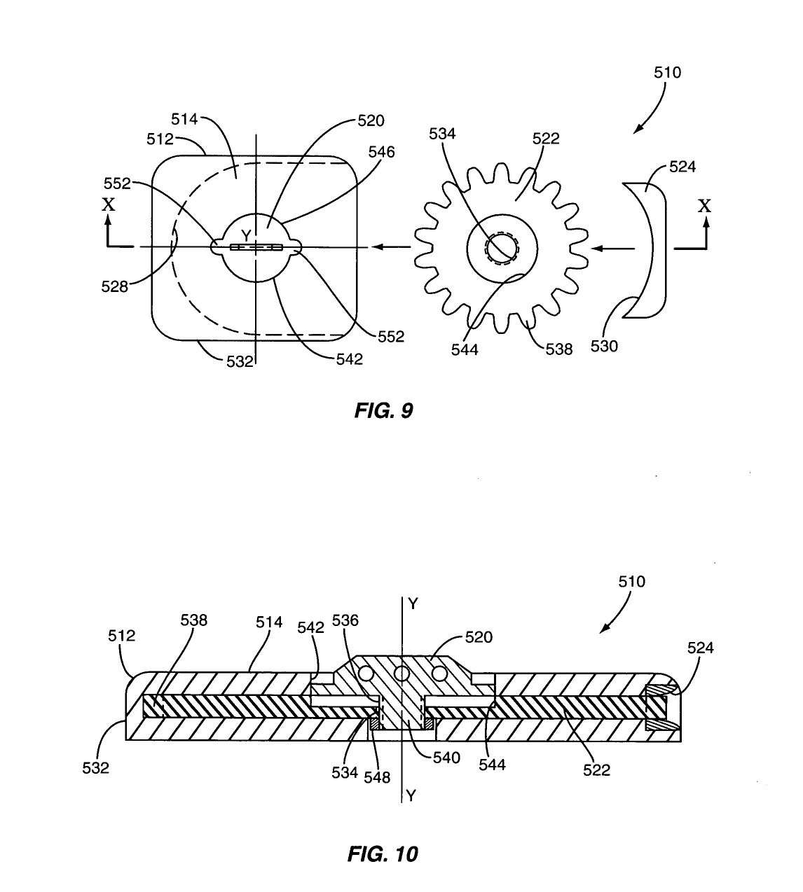

In embodiments described above, the gear member 122 and anchor member 120 move towards the extended position as the gear member 122 rotates about the threads 128, 136. In an embodiment of a vertebral implant 510 shown in FIGS. 9 and 10, the gear member 522 rotates within the implant body 512, but does not move axially as with previous embodiments. FIG. 9 depicts a top view of components of the exemplary vertebral implant 510 in an unassembled state. More specifically, FIG. 9 depicts a gear member 522, a cover 524, and an implant body 512 over which an anchor 520 is disposed. In the present embodiment, the gear member 522 is laterally inserted into a body 512 and into an inner cavity 528. The inner cavity 528 is open from a lateral side 532 of the body 512 to accept the gear member 522. The gear member 522 is retained within the cavity 528 by a cover 524 that is secured to the body 512 after the gear member 522 is inserted. The cover 524 includes an arcuate bearing surface 530 that cooperates with the cavity 528 to form a cylindrical void in which the gear member 522 rotates.

The gear member 522 includes gear teeth 538 as in previous embodiments, but the outer perimeter of the gear teeth 538 are not threaded. Instead, the gear member 522 includes an inner threaded aperture 534. The threaded aperture 534 is sized to accept a corresponding male thread 536 that is formed around the exterior of a post 540 that protrudes from the bottom of the anchor member 520. The cross section view shown in FIG. 10 more clearly illustrates the configuration of the body 512, gear member 522, and anchor 520. As shown, the threaded interface (534, 536) between the gear member 522 and anchor 520 tends to extend or retract the anchor 520 as the gear member 522 rotates within the implant body 512.

The anchor 520 is constrained to movement along axis X due in part to the fit between the perimeter 546 of the anchor 520 and aperture 542 in the implant body 512. Further, the illustrated anchor 520 is clocked with respect to the X axis by tabs 552. The illustrated gear member 522 also includes a recess 544 into which the anchor 520 sits when the anchor 520 is retracted. Thus, the recess 544 limits the extent to which the anchor 520 may be retracted. Correspondingly, a retainer 548 limits the extent to which the anchor 520 may be extended. In one embodiment, the retainer 548 is a threaded nut that is screwed onto the end of the post 540. In other embodiments, the retainer 548 is a clip or a snap ring.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. For instance, the vertebral implant (e.g., implant 110 from FIGS. 3, 4, and 5) disclosed herein may be implemented as endplates of a motion-preserving device with a nucleus disposed therebetween. In another implementation, the implant bodies may be disposed on opposite sides of an extendable device that is used in vertebrectomy or corpectomy procedures where one or more vertebral levels are removed. The present embodiments are, therefore, to be considered in all respects as illustrative and not restrictive, and all changes coming within the meaning and equivalency range of the appended claims are intended to be embraced therein.

What is claimed is:

1. An implant for insertion between vertebral members in a patient comprising:
a body including an outer surface and an aperture forming an inner cavity;
a gear rotatably disposed in the cavity, the gear including a rotation axis and a plurality of teeth; and
an anchor operatively coupled to the gear, the anchor including a bone-engaging feature to engage the vertebral members,
the gear and the anchor being movable substantially along the rotation axis between a first position in which the bone-engaging feature is retracted below the outer surface and a second position in which the bone-engaging feature is extended above the outer surface to contact one of the vertebral members.

2. The implant of claim 1 further comprising a locking mechanism that prevents movement of the bone engaging feature from the second position to the first position.

3. The implant of claim 1 wherein the anchor is disposed in sliding contact with the gear.

4. The implant of claim 1 wherein the anchor remains oriented in a predetermined position relative to the rotation axis when moving between the first and second positions.

5. The implant of claim 1 wherein the gear teeth are accessible from outside the body through a lateral opening in the body, gear teeth remaining accessible through the lateral opening when the gear is in the first and second positions.

6. The implant of claim 1 further comprising a cover disposed over the aperture, the cover including an aperture through which the bone engagement feature protrudes when the anchor is in the second position.

7. The implant of claim 6 wherein the cover maintains a rotational orientation of the anchor member while the anchor moves substantially along the rotation axis.

8. The implant of claim 1 further comprising a second bone engagement feature.

9. The implant of claim 1 further comprising a second gear operatively coupled to a second anchor including a second bone engagement feature, the second gear and the second anchor being movable substantially between a third position in which the bone-engaging feature is retracted below the outer surface and a fourth position in which the second bone-engaging feature is extended above the outer surface to contact one of the vertebral members.

10. An implant for insertion between vertebral members in a patient comprising:
a body including an outer surface and a threaded aperture forming an inner cavity, the threaded aperture defining a longitudinal axis;
a gear rotatably disposed in the threaded cavity, the gear including a plurality of teeth, the teeth including threads to mate with the threaded aperture; and
an anchor operatively coupled to the gear, the anchor including a bone-engaging feature to engage the vertebral members,
the rotation of the gear causing the anchor to move substantially along the longitudinal axis between a first position relative to the body and a second position relative to the body.

11. The implant of claim 10 wherein when the anchor is in the second position, the bone-engaging feature extends outside the body.

12. The implant of claim 10 wherein when the anchor is in the first position, the bone-engaging feature is retracted within the body.

13. The implant of claim 10 further comprising a locking mechanism that prevents movement of the bone engaging feature from the second position to the first position.

14. The implant of claim 10 wherein the anchor is disposed in sliding contact with the gear.

15. The implant of claim 10 wherein the anchor remains oriented in a predetermined rotational position relative to the longitudinal axis when moving between the first and second positions.

16. The implant of claim 10 wherein the rotation of the gear causes the gear to move along the threads and substantially along the longitudinal axis.

17. The implant of claim 16 wherein the gear teeth are accessible from outside the body through a lateral opening in the body, gear teeth remaining accessible through the lateral opening when the anchor is in the first and second positions.

18. The implant of claim 10 further comprising a cover disposed over the threaded aperture, the cover including a guiding aperture through which the bone engagement feature protrudes when the anchor is in the second position.

19. A method of inserting an implant between vertebral members in a patient, the method comprising the steps of:
positioning the implant between the vertebral members;
rotating a gear positioned within an implant body and causing the gear and an anchor within the implant body to move relative to the implant body;
maintaining a rotational orientation of the anchor while moving the gear and the anchor relative to the implant body; and
engaging the anchor with the vertebral members by further rotating the gear.

20. The method of claim 19 further comprising rotating the gear by engaging the teeth through an opening in the body.

21. The method of claim 20 wherein engaging the teeth through an opening in the body comprises engaging the teeth with an insertion tool that includes a rotating second gear.

22. The method of claim 19 further comprising locking the gear to prevent movement of the gear after engaging the anchor with the vertebral members.

23. The method of claim 19 wherein causing the gear and the anchor within the implant body to move relative to the implant body comprises pushing the anchor through contact between bearing surfaces on each of the anchor and the gear.

24. The method of claim 19 wherein causing the gear and the anchor within the implant body to move relative to the implant body comprises moving the gear along a threaded interface between the gear and the implant body.

25. The method of claim 19 wherein causing the gear and the anchor within the implant body to move relative to the implant body comprises moving the gear and the anchor along an axis about which the gear rotates.

26. A method of inserting an implant between vertebral members in a patient, the method comprising the steps of:
inserting the vertebral implant between the vertebral members, the vertebral implant including a body, a gear disposed within a threaded aperture in the body and rotatably engaged with the threaded aperture, and an anchor operatively coupled to the gear;
rotating the gear and moving the gear relative to the body and along the threaded aperture; and
moving the anchor between a first position in which the anchor is retracted within the body and a second position in which the anchor is extended outside of the body and in contact with the vertebral members.

27. The method of claim 26 further comprising rotating the gear by engaging the teeth through an opening in the body.

28. The method of claim 27 wherein engaging the teeth through an opening in the body comprises engaging the teeth with an insertion tool that includes a rotating second gear.

29. The method of claim 26 further comprising locking the gear to prevent movement of the gear along the threaded aperture after contacting the anchor with the vertebral members.

30. The method of claim 26 wherein moving the gear and the anchor along the threaded aperture further comprises pushing the anchor through contact between bearing surfaces on each of the anchor and the gear.

31. The method of claim 26 wherein moving the anchor along the threaded aperture further comprises maintaining a rotational orientation of the anchor.
